(19) United States
(12) Patent Application Publication
(10) Pub. No.: US 2022/0065866 A1
(43) Pub. Date: Mar. 3, 2022

(54) UTERINE ENDOMETRIAL FLUID FOR PREDICTION OF SUCCESS IN FERTILITY TREATMENT

(71) Applicant: Fertility Lab Sciences, LLC, LoneTree, CO (US)

(72) Inventors: Mandy Katz-Jaffe, Denver, CO (US); William B. Schoolcraft, Denver, CO (US)

(21) Appl. No.: 17/422,702

(22) PCT Filed: Jan. 24, 2020
(86) PCT No.: PCT/US20/15110

§ 371 Date: Jul. 13, 2021

Related U.S. Application Data
(60) Provisional application No. 62/796,695, filed on Jan. 25, 2019.
(60) Provisional application No. 62/841,008, filed on Apr. 30, 2019.

(51) Int. Cl. G01N 33/68; C12Q 1/6876

(52) U.S. Cl. CPC G01N 33/689; C12Q 1/6876; C12Q 2600/178; C12Q 2600/158; G01N 2800/367

(57) ABSTRACT

Provided herein are methods, systems, and kits for improving success rates in assisted reproductive technologies such as in vitro fertilization, frozen embryo transfer, and intrauterine insemination. These methods, systems, and kits rely on levels of protein, metabolite, and microRNA markers determined herein to be linked to uterine toxicity and embryo implantation failure.

### CROSS-REFERENCE TO RELATED APPLICATIONS

This application is a National Stage Application of PCT/US20/15110, filed Jan. 24, 2020, which claims priority under 35 U.S.C 119 (e) to U.S. Provisional Patent Application Ser. No. 62/796,695, entitled “Uterine Endometrial Fluid for Prediction of Success in Fertility Treatment,” filed Jan. 25, 2019 and to U.S. Provisional Patent Application Ser. No. 62/841,008, entitled “Uterine Endometrial Fluid for Prediction of Success in Fertility Treatment,” filed Apr. 30, 2019, each of which are incorporated herein by reference. To the extent appropriate, a claim of priority is made to each of the above-disclosed applications.

### FIELD

The invention relates to the field of fertility treatment. More particularly, evaluation of a uterine microenvironment is used to enhance the pregnancy success rate, for example, when a fertilized egg is implanted into a patient's uterus. In addition, manipulation of the uterine microenvironment improves the chance of pregnancy in both natural and artificial cycles.

### DESCRIPTION OF THE RELATED ART

Despite progressively improving IVF pregnancy rates, the majority of transferred human embryos result in implantation failure. For example, the Society for Assisted Reproductive Technologies (to which about 80% of U.S. fertility clinics report) reports IVF success rates in 2015 (% patients with live births using their own eggs) at 56.7% for women younger than 35 years old, 44.4% for women aged 35-37, 30.7% for women aged 38-40, 15.1% for women aged 41-42, and 4.5% for women older than 42 years old. Clearly, implantation success rates decrease with the increasing maternal age if donor eggs are not used. Various factors are associated with implantation failure, including embryo chromosome aneuploidies related to advanced maternal age and maternal factors such as endometrium response failure to hormone regulation.

Various approaches are typically taken to overcome low implantation success rates. In the past, and still practiced at some clinics, multiple embryos are transferred during a single IVF procedure to improve odds of implantation. The process for selecting embryos for transfer often involves grading methods developed in individual laboratories to judge oocyte and embryo quality. An arbitrary embryo score, involving the number and quality of embryos, may reveal the probability of pregnancy success post-transfer. For example, an embryologist may grade embryos using morphological qualities including the number of cells, clearness of cytoplasm, evenness of growth and degree of fragmentation. Often, several embryos selected for these general qualities are implanted to improve the chance of pregnancy. However, embryo selection based on morphological qualities is not precise—for example, morphological evaluation fails to evaluate two factors related to embryo viability: chromosomal integrity and embryo metabolism. In clinics that perform morphological screening of embryos, the number of embryos transferred depends upon the number of viable embryos available, the age of the woman and other health and diagnostic factors.

The transfer of multiple embryos, however, often results in multiple pregnancies, a major complication of IVF. In general, multiple pregnancies, specifically, more than twins, hold maternal and fetal risks. For example, multiple births are associated with increased risk of pregnancy loss, neonatal morbidity, obstetrical complications, and prematurity with potential for long term damage. Some countries implement strict limits on the number of transferred embryos to reduce the risk of high-order multiples (e.g., triplets or more), and the American Society of Reproductive Medicine has its own guidelines for the number of embryos to transfer. However, these limitations are not universally followed or accepted.

In parallel with embryo preparation, the uterine environment is prepared for reception of the embryo(s) by hormonal manipulation of the female patient such that both the embryo and the patient are ready for the embryo transfer at the same time. Hormonal manipulation involves administration of estrogen and progesterone at levels required to mimic or exceed the circulating blood levels of those hormones in a normal woman near the end of a typical monthly cycle.

Commercial endometrial receptivity tests are offered to infertility patients for testing 30-90 days prior to embryo transfer, but the results are not necessarily indicative of the uterine environment at the time of embryo transfer. Though endometrial lining thickness is measured by ultrasound just prior to frozen embryo transfer, and blood reproductive hormones are monitored, as of yet, there is no way of testing whether a woman's uterus will become receptive in a current cycle. In order to improve fertility treatment success rates and to assist women with endometrial-based infertility, methods of identifying receptive uterine endometrium in a patient are needed.

### SUMMARY

Provided herein are methods, systems, and kits for obtaining, using, and/or analyzing data associated with improving fertility treatment success rates. In some aspects, the data is obtained by non-invasively sampling a patient's uterine microenvironment for the current treatment cycle, i.e. the cycle coinciding with the scheduled embryo transfer or intrauterine insemination, not a cycle several months prior to a scheduled procedure.

As such, provided herein is a method of predicting negative pregnancy outcome in fertility treatment. In some embodiments, the method comprises: (a) contacting uterine endometrial secretions from a patient with a kit that comprises a solid-state substrate functionalized to identify at least two markers associated with hostile endometrial environment selected from the group consisting of proteins, metabolites, or miRNAs, and (b) determining the secretome profile of the uterine endometrial secretions to ascertain an increase or decrease in the presence of markers associated with a hostile endometrial environment. An increase or decrease in the presence of the one or more markers associated with a hostile endometrial environment, relative to the secretome profile of uterine endometrial secretions of a successful pregnancy outcome, predicts negative pregnancy outcome.

Provided herein is a kit comprising a solid-state substrate functionalized to identify one or more markers, or at least two markers, associated with a hostile endometrial environment selected from the group consisting of proteins, metabolites, and miRNAs. The kit can comprise an immunosorbent assay, instructions on how to perform the assay, a model for classifying the data obtained from the assay, and/or a secretome profile of uterine endometrial secretions associated with a successful pregnancy outcome.

Also provided herein is a system for enhancing the pregnancy success rate of fertility treatment. In some embodiments, the system comprises the step of predicting a negative pregnancy outcome in a patient undergoing fertility treatment prior to frozen embryo transfer or intrauterine insemination. The step of predicting comprises determining the secretome profile of the patient's uterine endometrial secretions to ascertain an increase or decrease in the presence of markers associated with a hostile endometrial environment. An increase or decrease in the presence of one or more markers associated with a hostile endometrial environment, relative to the secretome profile of uterine endometrial secretions associated with a successful pregnancy outcome, predicts negative pregnancy outcome in the patient.

In some aspects, the step of determining the secretome profile of uterine endometrial secretions comprises contacting uterine endometrial secretions from a patient with a kit that comprises a solid-state substrate functionalized to identify at least two markers associated with a hostile endometrial environment selected from the group consisting of proteins, metabolites, or miRNAs, and determining the secretome profile of the uterine endometrial secretions to ascertain an increase or decrease in the presence of markers associated with a hostile endometrial environment.

Provided herein is an in vitro method of screening a fertility patient prior to frozen embryo transfer or intrauterine insemination. In some embodiments, the method comprises (a) contacting uterine endometrial secretions from the patient with a kit that comprises a solid-state substrate functionalized to identify at least two markers associated with a hostile endometrial environment selected from the group consisting of proteins, metabolites, or miRNAs, and (b) determining the secretome profile of the uterine endometrial secretions to ascertain an increase or decrease in the presence of markers associated with a hostile endometrial environment. An increase or decrease in the presence of the one or more markers associated with a hostile endometrial environment, relative to the secretome profile of uterine endometrial secretions of a successful pregnancy outcome, predicts negative pregnancy outcome.

In some embodiments, a method of predicting implantation failure of a candidate embryo is provided. The method comprises (a) contacting uterine endometrial secretions from a patient with a kit that comprises a solid-state substrate functionalized to identify at least two markers associated with a hostile endometrial environment selected from the group consisting of proteins, metabolites, or miRNAs, and (b) determining the secretome profile of the uterine endometrial secretions to ascertain an increase or decrease in the presence of markers associated with a hostile endometrial environment. The increase or decrease in the presence of one or more markers associated with a hostile endometrial environment, relative to the secretome profile of uterine endometrial secretions of a successful pregnancy outcome, predicts embryo implantation failure.

In some aspects the marker is a protein. In some embodiments, the protein is selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment. In some embodiments, the protein is selected from the group consisting of SOD1, PRDX6, PLA2G4D, and TET1, wherein decreased expression of the protein is associated with a hostile endometrial environment.

In some embodiments, the marker is arginine, wherein decreased levels of arginine is associated with a hostile endometrial environment.

In some aspects, the marker is a microRNA. In some embodiments, the microRNA is selected from the group consisting of hsa-miR-891a, hsa-miR-522, hsa-miR-198, and hsa-miR-365, and decreased presence is associated with a hostile endometrial environment. In some embodiments, the microRNA is selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and increased presence is associated with a hostile endometrial environment.

In some aspects, the marker is a metabolite. In some embodiments, the metabolite is selected from the group consisting of xanthine, docosahexaenoic acid, fumarate, cysteine, putrescine, proline, leucine/isoleucine, hypoxanthine, alanine, adenosine, 8z-11z-14z-icosatrienoic acid, 8z-11z-14z-17z-icosapentaenoic acid, and 5-oxoproline, and decreased presence of the metabolite is associated with a hostile endometrial environment. In some embodiments, the metabolite is selected from the group consisting of urate, citrate, orthophosphate, and heptanoic acid, and increased presence of the metabolite is associated with a hostile endometrial environment.

In still other aspects, a patient's uterine microenvironment is observed and negative implantation outcomes 24 hours prior to an embryo transfer are assessed. An interplay of several biological processes can be evident in aspirates from a uterine microenvironment predicted to experience a failed transfer: in particular, 13 reduced transcripts, 7 increased maternal miRNAs, 12 decreased amino acids, and 16 proteins of altered abundance. In some aspects, a decreased expression of PLA2G4D which regulates the eicosanoid pathway, thereby impacting downstream synthesis of prostaglandins like PGE2, can be predictive of a failed transfer. In some aspects, decreased expression of TET1, an epigenetic regulator required for DNA methylation, can be predictive of a failed transfer. In some aspects, increased levels of miR-17, a known negative regulator of VEGFA, which is required for successful implantation, can be predictive of a failed transfer. In some aspects, decreased quantities of arginine, essential for blastocyst activation and trophectoderm motility, can be predictive of a failed transfer. Lastly, an increased abundance of SERPING1, a protein associated with inflammation, which regulates complement activation, can be predictive of a failed transfer.

In one embodiment, a system is provided for enhancing the pregnancy success rate for a fertility patient. The system includes an electronic system configured to gather uterine endometrial secretome data as a secretome profile by quantitating markers implicated in uterine toxicity. A model is provided for use in recommending whether to implant an embryo or perform an intrauterine insemination on the basis of this secretome profile. The secretome data may be, for example, obtained by use of mass spectroscopy, qPCR, or ELISA measurements.

According to one aspect of the system, the uterine secretome profile may be provided by identifying markers in uterine endometrial secretions that may be linked to changed odds of implantation success, for example, microRNAs, metabolites, and proteins.

In one embodiment, a method of fertility treatment entails determining the uterine endometrial secretome profile of a fertility patient where the profile is generated by measuring markers implicated in uterine toxicity. This provides data that may be submitted to a model that associates one or more of these markers with changes in odds of embryo implantation success or failure. A recommendation for implantation of the embryo or intrauterine insemination may then be provided based upon the modeling outcome. The embryo may be conditionally implanted on the basis of the recommendation.

In one embodiment, there is an improved ELISA kit with a plurality of microwells for the quantitation of protein content in a sample. The microwells are constructed and arranged to quantitate for a plurality of proteins implicated in uterine toxicity.

### BRIEF DESCRIPTION OF THE FIGURES

FIG. 1 shows a PCA plot separating positive and negative uterine samples in regards to their metabolite profile.

FIG. 2 specifies the differentially expressed metabolites.

FIGS. 3, 4 and 5 provide box plots for each differentially expressed individual metabolite.

FIG. 6 shows the 3 cytokines (out of 30 that were analyzed) that were differentially expressed.

### DETAILED DESCRIPTION

The following definitions are provided to facilitate understanding of certain terms used herein and are not meant to limit the scope of the present disclosure.

The phrase “negative pregnancy outcome” refers to the failure of an embryo to implant in a woman's uterus, or the failure of an embryo to implant sufficiently to maintain the life of the embryo. This phrase can be used interchangeably with the phrase “implantation failure”.

“Implantation failure” occurs when an otherwise favorable embryo fails to implant, and repeated implantation failure may be designated when otherwise favorable embryos fail to implant after several IVF treatment attempts.

The phrase “hostile endometrial environment” refers to an environment in the uterus which compromises implantation of an embryo. Particularly, the microenvironment of the endometrium where the embryo implants can lack a suitable support system for an implanting embryo, such that implantation of the embryo ultimately fails resulting in a negative pregnancy outcome. Uterine a hostile endometrial environment can be related to inflammation, as a result of auto-immunity, for example, or external inflammatory inputs, or can be related to oxidative stress or the body's inability to address oxidative stress.

The window of implantation, which occurs naturally around day 6-10 post ovulation, is a period of endometrial receptivity. It is short and results from the programmed sequence of estrogen and progesterone on the endometrium. This is a critical time point when the embryo and endometrium encounter each other and exchange molecular dialogue for the first time. Within the architecture of the endometrium, specific property changes in adhesion need to occur to allow blastocyst attachment, as well as tight regulation of signaling pathways in the surrounding microenvironment. Successful molecular interchange between the embryo and a receptive endometrium must occur during the initial stages of implantation. Implantation is characterized by structural and functional changes in the endometrial layer and secretion of nutrients, including numerous vitamins and steroid-dependent proteins. Without this molecular exchange, blastocyst adhesion to a receptive uterus will be unsuccessful.

Endometrial aspirations at the time of embryo transfer using a transfer catheter are an effective, minimally invasive means of sampling the endometrial micro-environment in localized areas of the uterus. Endometrial aspirates collected 24 hours prior to embryo transfer contained specific prostaglandin levels that were correlated with successful implantation, and collection of the aspirate itself had no negative impact on pregnancy outcome (Vilella et al, 2013). Other commercially available technologies offering insight into the window of implantation rely on tissue from endometrial biopsies. However, these samples are obtained from previous cycles, which may not represent the molecular state of the current cycle.

Provided herein are methods, kits, and systems which allow a rare glimpse into the microenvironment a transferred embryo will encounter. This sampling technique permits identification of patient etiologies that may require additional medical intervention in order to achieve a successful blastocyst implantation.

One of the more difficult patient populations to treat in ART are those presenting with repeat implantation failure. Repeat implantation failure is defined as cases in which women have 3 or more failed embryo transfer attempts with euploid embryos. There are many known factors that may contribute to implantation failure, including maternal factors such as immunologic factors and impaired endometrial function, in addition embryonic factors including genetic abnormalities.

However, in patient populations with euploid embryos that have already been screened and appropriately treated for known maternal factors, there remain a subset with no clear cause of impaired endometrial function. For these patients, molecular factors affecting their endometrial microenvironment are predictive of a successful or failed embryo transfer in a given cycle. Endometrial aspirations, when combined with OMICS technologies, provide insights into the receptive state during the window of implantation, and permit identification of factors potentially involved in infertility.

Developing an ideal environment for blastocyst implantation is thought to require interplay between the immune and endocrine systems. A receptive endometrium permits the invasion of the blastocyst and the rapid growth of the placenta while supporting the transformation of uterine cells into decidual cells. This can be facilitated by immune cells already present in the uterus, the cytokines secreted by those immune cells, and hormonal changes.

The most intensively studied aspects of the uterine microenvironment are the uterine immune cells: maternal CD4+CD25+ Foxp3+ regulatory T cells (Tregs), uterine natural killer cells (uNK cells), uterine dendritic cells (uDCs), uterine mast cells (uMCs), and uterine macrophages. However, very little is known about the rest of the uterine microenvironment that contributes to successful blastocyst implantation.

Provided herein are methods, systems, and kits for enhancing the pregnancy success rate, for example, in fertility treatment involving embryo transfer or intrauterine insemination, or regulation of ovulation or inducing ovulation. Also provided herein are methods of treating female infertility comprising: (a) ascertaining miR-17 levels in the uterine endometrial secretions from a patient, (b) treating a patient having an increase in miR-17 levels in the uterine endometrial secretions relative to a miR-17 profile indicative of a successful pregnancy outcome with recombinant human VEGF-A; and (c) performing frozen embryo transfer or intrauterine insemination. In some aspects, (a) is performed at least 24 hours prior to (c). Additional methods of treatment are contemplated wherein the uterine marker is ascertained and if decreased, the protein, amino acid, etc., can be administered to the patient in an amount sufficient to increase the level to provide an endometrial environment suitable for implantation.

In some embodiments, a method of predicting negative pregnancy outcome in fertility treatment is provided. Application of this method permits a health care provider to identify a non-receptive uterus in a fertility patient, prior to performing frozen embryo transfer or intrauterine insemination. In some aspects the method comprises (a) contacting uterine endometrial secretions from the patient with a kit that comprises a solid-state substrate functionalized to identify at least two markers associated with a hostile endometrial environment, and (b) determining the secretome profile of the uterine endometrial secretions to ascertain an increase or decrease in the presence of markers associated with a hostile endometrial environment. The secretome profile of the patient's uterine endometrial secretions, relative to the secretome profile of uterine endometrial secretions of a successful pregnancy outcome, can indicate a toxic uterine environment. The secretome profile of the patient's uterine endometrial secretions, relative to the secretome profile of uterine endometrial secretions of a successful pregnancy outcome, can predict a successful implantation or a negative pregnancy outcome.

Inflammatory markers related to a hostile endometrial environment include pro/anti-inflammatory mediators, complement regulators, and/or proteins involved in chemotaxis. In some aspects, uterine endometrial secretions may contain increased levels of any one or more of ITIH4, LTF, SERPING1, GC, CHF, and/or CP.

Markers of oxidative stress related to a hostile endometrial environment include pro/anti-oxidative effects, loss of antioxidants, and loss of the body's ability to deal with reactive oxidative species. In some aspects, uterine endometrial secretions may contain increased levels of any one or more of GGT1, LTF, or CFH. In some aspects, uterine endometrial secretions may contain decreased levels of SOD1 and/or PRDX6.

Markers involved with implantation related to a hostile endometrial environment include TGFB sequestration, trophoblast invasion, ECM remodeling, and uterine receptivity. In some aspects, uterine endometrial secretions may contain increased levels of any one or more of THSD4, ANPEP, COL6A1, PROM1, ITIH4, and PLG. In some aspects, uterine endometrial secretions may contain decreased levels of SOD1 and/or PRDX6.

In some aspects, the patient's uterine endometrial secretions are obtained prior to thawing of the embryo for transfer. For example, the endometrial secretions can be obtained within 48 hours of a scheduled transfer, or within 36 hours of a scheduled transfer, or within 24 hours of a scheduled transfer, or within 18 hours of a scheduled transfer, or within 12 hours of a scheduled transfer. Typically, an embryo is thawed one hour prior to the embryo transfer, and the data for the secretome profile can be obtained any time prior to thawing. The sample of the uterine endometrial secretions is obtained in a non-invasive manner, i.e. no biopsy is needed. In some aspects, the sample is obtained by catheter.

By ascertaining the health of the patient's uterine microenvironment prior to thawing of the embryo, a decision can be made to defer the embryo transfer procedure to a later cycle. This saves the embryo from the thaw cycle preserving viable embryos for later transfer, a benefit to the methods and systems provided herein. Another benefit to this process is that the patient's uterine microenvironment is assessed for the current cycle, not a cycle several months prior to a scheduled transfer. This is important as the condition of a fertility patient's endometrium can change from cycle to cycle.

Various markers are provided herein that have been associated with a negative pregnancy outcome. In some aspects, the marker is a cytokine, for example, IL-6, IL-8, VEGF. In some aspects, the marker is a mucin protein, for example, Mucin-1, Mucin-16, Mucin-5B, or Mucin-5AC. In some aspects, the marker is selected from the group consisting of an IgGFc-binding protein, Carbonic anhydrase 1, or Cystatin-C, ITIH4, LTF, SERPING1, GC, CFH, FFT1, THSD4, ANPEP, COL6A1, PROM1, and PLG. In each instance, increased expression of the protein is associated with a hostile endometrial environment.

In some aspects, the marker is a protein selected from the group consisting of SOD1, PRDX6, PLA2G4D, and TET1, wherein decreased expression of the protein is associated with a hostile endometrial environment.

In some aspects, the marker is arginine, wherein decreased levels of arginine is associated with a hostile endometrial environment.

In some aspects, the marker is PLA2G4D, a member of the phospholipase A2 enzyme family. This protein catalyzes the hydrolysis of glycerophospholipids at the sn-2 position, liberating free fatty acids and lysophospholipids. PLA2G4D regulates the eicosanoid pathway, impacting downstream synthesis of prostaglandins responsible for Wnt signaling activation. Decreased PLA2G4D expression is indicative of a hostile endometrial environment.

In some aspects, the marker is TET1. TET1 regulates numerous genes defining cellular differentiation. In epiblast cells, TET1 demethylates gene promoters via hydroxymethylation and maintains telomere stability. Decreased TET1 expression has also been correlated with endometrial tumor progression. Decreased TET1 expression is indicative of a hostile endometrial environment.

MicroRNAs (miRNAs) are short, non-coding regulatory RNAs that are an integral component in the regulation of protein expression. MicroRNAs contribute to endometrial embryo crosstalk and are essential for successful implantation. In some aspects, the marker is a microRNA, for example, hsa-miR-891a, hsa-miR-522, hsa-miR-198, or hsa-miR-365. Decreased presence of the microRNA is associated with a hostile endometrial environment. In other aspects, the microRNA is hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, or hsa-miR-106c. Increased presence of the marker is associated with a hostile endometrial environment.

The miR-17/92 cluster collectively targets thousands of genes, and is involved in many cellular processes in both the adult organism and the developing embryo. The target genes of has-miR-17-5p genes are involved in many cellular processes, including cell growth, cell differentiation, apoptosis, and cellular homeostasis. miR-17 inhibits VEGFA, causing decreased cell proliferation, migration, and adhesion. Recombinant VEGF-A significantly increased endometrial epithelial cell adhesion. The VEGF-A protein specifically acts on acts on endothelial cells and has various effects, including mediating vascular permeability, angiogenesis, cell growth, cell migration, and inhibiting apoptosis. In some aspects, greater than 2 fold increase in miR-17 is associated with a hostile endometrial environment.

In some aspects, the marker is an amino acid, for example, arginine. Arginine is required for survival, growth, and development of conceptuses during the peri implantation period. In embryos, it is critical for cell proliferation. Altered arginine expression involved in exaggerated inflammatory response and vascular dysfunction associated with poor endometrial receptivity and recurrent spontaneous miscarriage. A decrease in arginine in the uterine environment may impact implantation due to a lack of motility. Altered arginine expression may also be involved in the mechanism of exaggerated inflammatory response and vascular dysfunction associated with poor endometrial receptivity in women with recurrent spontaneous miscarriage (Banerjee et al. 2014).

Interestingly a decrease in arginine in the uterine environment may further impact implantation not because a lack of adhesion, but rather the lack of motility (Gonzalez et al. 2012), preventing the blastocyst from implanting at an appropriate implantation site.

In some aspects, the marker is a metabolite, for example, urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline. Decreased presence of the marker is associated with a hostile endometrial environment.

In accordance with the methods, systems, and kits provided herein, the markers can be used alone or in combination. For example, it is contemplated herein that a method, system, or kit might utilize any one or more of IL-6, IL-8, VEGF, Mucin-1, Mucin-16, Mucin-5B, Mucin-5AC, an IgGFc-binding protein, Carbonic anhydrase 1, Cystatin-C, hsa-miR-891a, hsa-miR-522, hsa-miR-198, hsa-miR-365, hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, or hsa-miR-106c, arginine, urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline in generating a uterine secretome profile.

In some embodiments, a kit is provided comprising a solid-state substrate functionalized to identify one or more, or at least two, markers associated with a hostile endometrial environment. The markers can be selected from the group consisting of proteins, metabolites, and miRNAs. In some aspects, the kit comprises an immunosorbent assay, a qPCR assay, instructions on how to perform the assay, a model for classifying the data obtained from the assay, and/or a secretome profile of uterine endometrial secretions associated with a successful pregnancy outcome.

In some embodiments, a system for enhancing the pregnancy success rate of fertility treatment is provided. In some aspects, the system comprises predicting a negative pregnancy outcome in a patient undergoing fertility treatment prior to frozen embryo transfer or intrauterine insemination. The step of predicting can comprise determining the secretome profile of the patient's uterine endometrial secretions to ascertain an increase or decrease in the presence of markers associated with a hostile endometrial environment. An increase or decrease in the presence of one or more markers associated with a hostile endometrial environment, relative to the secretome profile of uterine endometrial secretions of a successful pregnancy outcome, predicts negative pregnancy outcome in the patient.

In some aspects, the step of determining the secretome profile of uterine endometrial secretions comprises contacting uterine endometrial secretions from a patient with a kit that comprises a solid-state substrate functionalized to identify at least two markers associated with a hostile endometrial environment selected from the group consisting of proteins, metabolites, or miRNAs, and determining the secretome profile of the uterine endometrial secretions to ascertain an increase or decrease in the presence of markers associated with a hostile endometrial environment.

The secretome profile can be generated by data obtained from any method known to one of skill in the art to identify proteins, metabolites, or microRNAs. For example, the data can be obtained by mass spectroscopy, by an immunosorbent assay (for example, Enzyme-Linked Immunosorbent Assay (ELISA)), or by qPCR.

An in vitro method of screening a fertility patient prior to frozen embryo transfer or intrauterine insemination is also provided herein. In some aspects, the method comprises contacting uterine endometrial secretions from a patient with a kit that comprises a solid-state substrate functionalized to identify one or more, or at least two, markers associated with a hostile endometrial environment selected from the group consisting of proteins, metabolites, or miRNAs. The method further comprises determining the secretome profile of the uterine endometrial secretions to ascertain an increase or decrease in the presence of markers associated with a hostile endometrial environment. The increase or decrease in the presence of one or more markers associated with a hostile endometrial environment, relative to the secretome profile of uterine endometrial secretions of a successful pregnancy outcome, predicts a negative pregnancy outcome in the fertility patient. With a negative pregnancy outcome prediction, a caregiver can recommend forgoing the frozen embryo transfer procedure or the intrauterine insemination. Steps can be taken to reduce a hostile endometrial environment, including treatment to address the marker(s) associated with that particular patient's a hostile endometrial environment. For example, a patient might be treated with recombinant VEGF-A if VEGF protein expression in the patient uterine endometrial secretions is decreased relative to VEGF expression profile indicative of a successful pregnancy outcome. Likewise, a patient might be treated with recombinant VEGF-A if miR-17 levels are increased in the patient uterine endometrial secretions relative to miR-17 levels indicative of a successful pregnancy outcome.

Provided herein are methods of treating female infertility. In some aspects the method comprises: (a) ascertaining miR-17 levels in the uterine endometrial secretions from a patient, (b) treating a patient having an increase in miR-17 levels in the uterine endometrial secretions relative to a miR-17 profile indicative of a successful pregnancy outcome with recombinant human VEGF-A; and (c) performing frozen embryo transfer or intrauterine insemination. In some aspects, step (a) is performed at least 24 hours prior to step (c).

A method of predicting implantation failure of a candidate embryo is provided herein. In some embodiments, the method comprises: contacting uterine endometrial secretions from a patient with a kit that comprises a solid-state substrate functionalized to identify one or more, or at least two, markers associated with a hostile endometrial environment selected from the group consisting of proteins, metabolites, or miRNAs. The method further comprises determining the secretome profile of the uterine endometrial secretions to ascertain an increase or decrease in the presence of markers associated with a hostile endometrial environment. An increase or decrease in the presence of one or more markers associated with a hostile endometrial environment, relative to the secretome profile of uterine endometrial secretions of a successful pregnancy outcome, predicts embryo implantation failure.

### Example 1: Maternal Endometrial Secretions 24 Hours Prior to Frozen Embryo Transfer is Predictive of Implantation Outcome

Objective: Successful implantation can be dependent on the intricate dialogue between a competent embryo and a receptive endometrium. On the maternal side, specific biological changes in adhesion need to occur for blastocyst attachment, while tight regulation of signaling pathways are crucial for the invading embryo. The objective of this study was to examine the uterine fluid milieu in association with implantation outcome 24 hours prior to, and at the time of euploid embryo transfer.

Materials and methods: Infertile patients (n=48) were recruited with IRB consent prior to an estradiol/progesterone replacement frozen embryo transfer (FET) with euploid blastocysts. Uterine secretions were collected by gentle aspiration (˜2-5 ul), either 24 h prior to, or at the time of FET. In brief, using the mock transfer protocol typically performed prior to an embryo transfer, the tip of an empty embryo transfer catheter, covered by the protective sheath to avoid cervical mucus contamination, was positioned near the site where an embryo transfer would occur. After pulling away from the site slightly, so as not to disturb the potential implantation site, a small amount of uterine mucus was aspirated into a Leur-Lok 10 mL syringe (about 5-10 ul of uterine mucus). The catheter was then withdrawn, still taking care to avoid cervical mucus contamination. The tip of the catheter containing the aspirate was inserted into a dolphin nose 2 mL microtube and, using sterile scissors, the tip was cut off into the tube and flash frozen in liquid nitrogen. The samples were stored at −80° C. until further analysis.

Uterine secretome analysis was performed blinded of implantation outcome using qPCR for miRNA analysis (n=12) and mass spectrometry (n=36) for metabolite analysis (UHPLS-MS, Thermo) and protein analysis (LC-MS/MS, Thermo). MiRNA profiles were analyzed by REST® statistical software. MS data was converted with MassMatrix and processed with Maven (Princeton Univ). MS/MS data was examined using Mascot™ (v 2.2) and Scaffold (v 2.06). Validation of target genes was performed using qPCR on endometrial biopsies (n=14) and surplus cryopreserved blastocysts (n=14) donated with patient consent.

Results: A notable uterine secretome profile of miRNA, metabolites and proteins was significantly associated with a negative, toxic environment both 24 hours prior to, and at the time of embryo transfer (P<0.05, >2 fold change).

Specifically, several maternal miRNAs showed decreased expression with negative implantation, and several miRNAs showed increased expression with negative implantation, including miR-17 (P<0.05). See Tables 1 and 2. A known target gene of miR-17 through negative regulation is VEGFA, a signal protein essential for implantation and secreted by the receiving endometrium as well as the implanting embryo. Validation of VEGFA expression was confirmed in epithelial endometrial cells and individual blastocysts.

TABLE 1
microRNAs having decreased expression
associated with negative implantation

| | Negative |
|---|---|
| miRNA | Implantation |
| hsa-miR-891a | Decreased Expression |
| hsa-miR-522 | Decreased Expression |
| hsa-miR-198 | Decreased Expression |
| hsa-miR-365 | Decreased Expression |

TABLE 2
microRNAs having increased expression
associated with negative implantation

| | Negative |
|---|---|
| miRNA | Implantation |
| hsa-miR-135a | Increased Expression |
| hsa-miR-17 | Increased Expression |
| hsa-miR-10b | Increased Expression |
| hsa-miR-126 | Increased Expression |
| hsa-miR-155 | Increased Expression |
| hsa-miR-19a | Increased Expression |
| hsa-miR-150 | Increased Expression |
| hsa-miR-200c | Increased Expression |
| hsa-miR-224 | Increased Expression |
| hsa-miR-140 | Increased Expression |
| hsa-miR-222 | Increased Expression |
| hsa-miR-31 | Increased Expression |
| hsa-miR-454 | Increased Expression |
| hsa-miR-106c | Increased Expression |

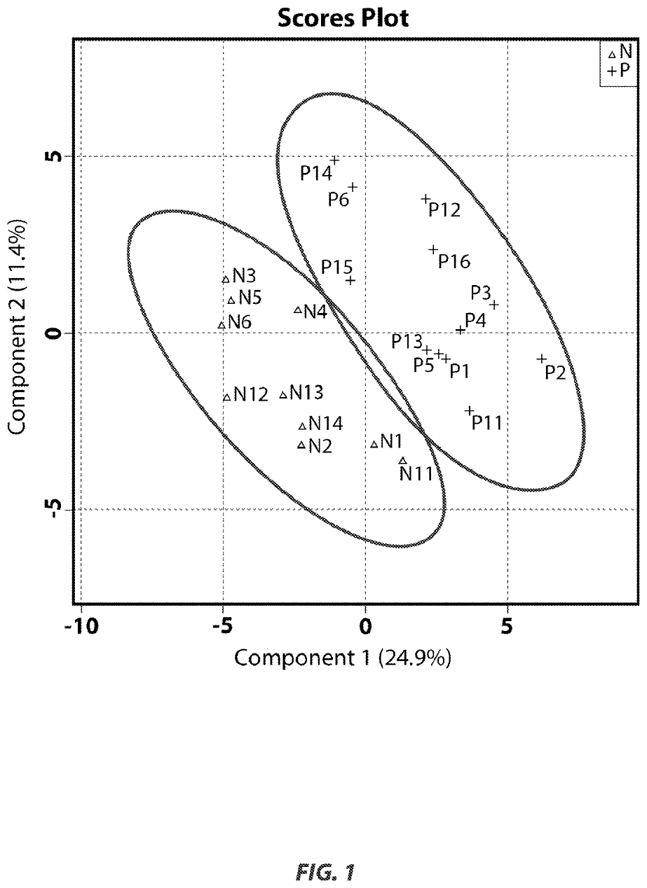

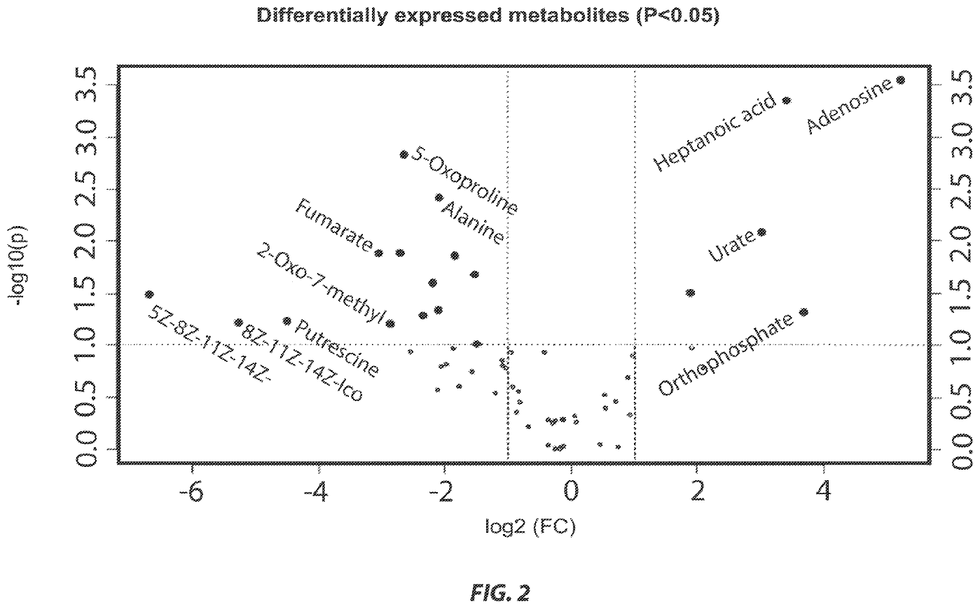

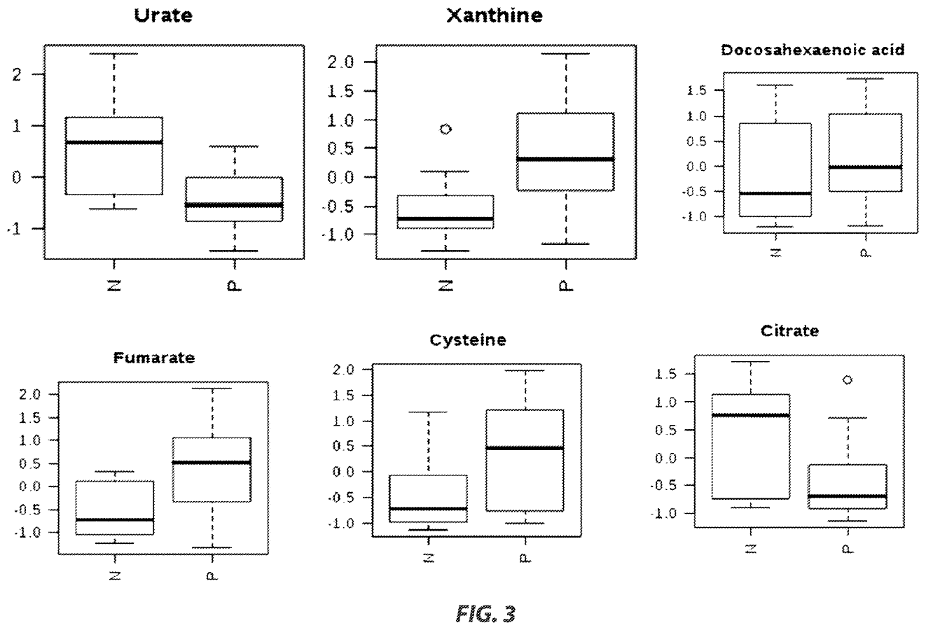

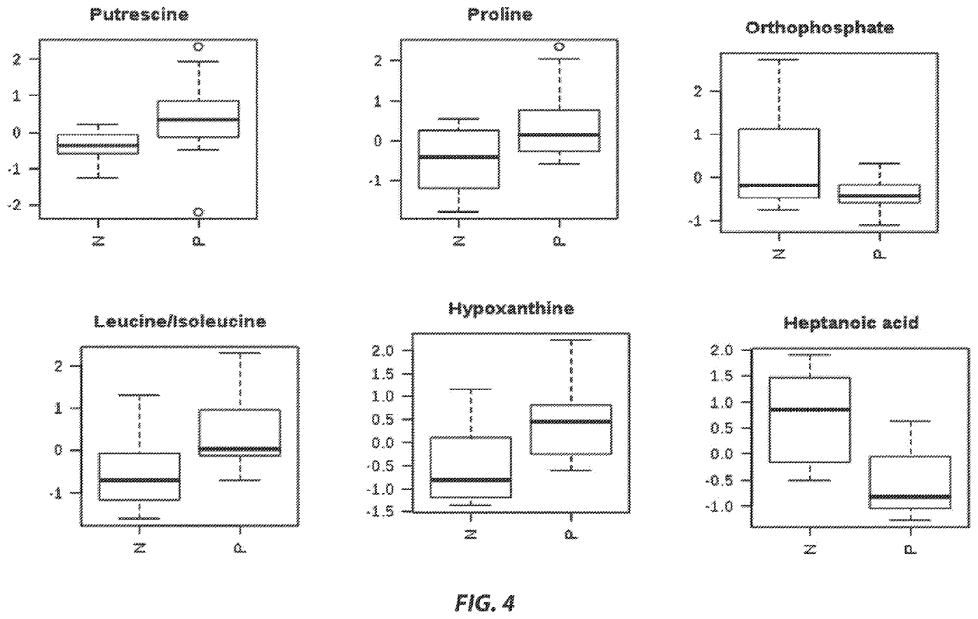

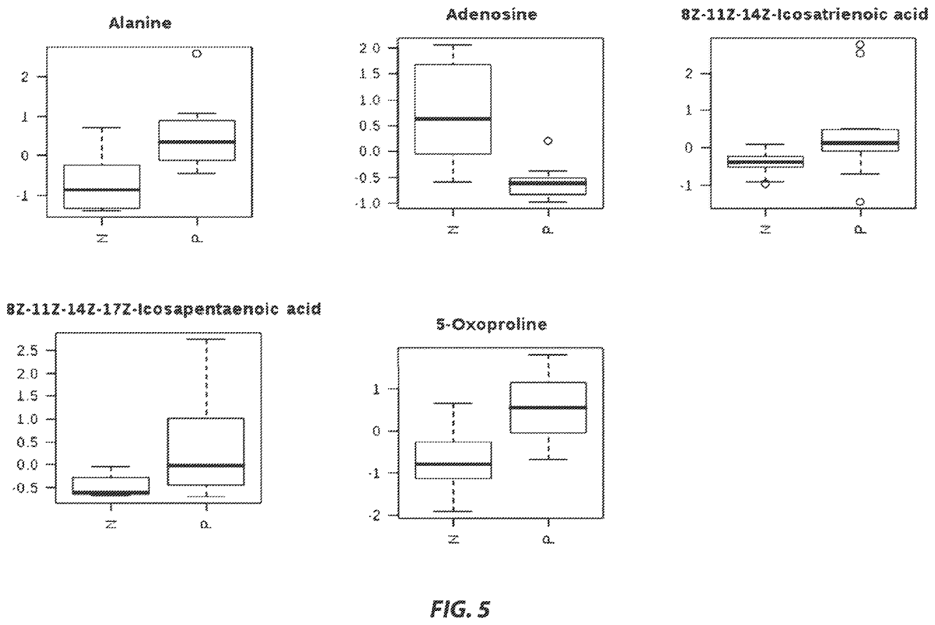

A total of 17 metabolites displayed significant decreased quantities in the uterine secretome associated with negative implantation (P<0.05, >2 fold change) including arginine, essential for blastocyst activation and trophectoderm motility, and urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline. See FIGS. 1-5.

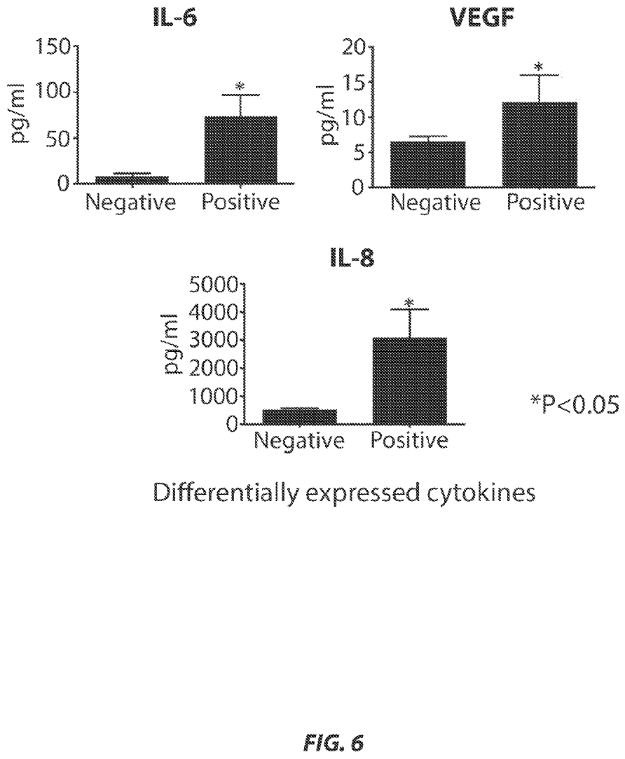

Three cytokines, VEGF, IL-6 and IL-8, out of 30 tested were associated with negative implantation. See FIG. 6. Cytokines were identified by ELISA.

A total of 469 proteins were screened by LC-MS/MS. Seven proteins had increased expression associated with a negative pregnancy outcome (P<0.05): Mucin-1, Mucin-16, Mucin-5B, Mucin-5AC, IgGFc-binding protein, Carbonic anhydrase 1 and Cystatin-C. Mucins are glycosylated epithelial cell surface proteins that have considerable effect on endometrial function, creating a barrier to implantation. Overexpression of mucin proteins is associated with maintaining a non-receptive uterine surface.

Conclusion: Aberrant maternal uterine miRNA and molecular secretions allow for the characterization of implantation failure both 24 hours prior to, and at the time of FET. This compromised embryo-endometrial dialogue further impacts the transcription levels of key signaling molecules, resulting in significantly lower implantation success. Predicting the maternal molecular microenvironment ahead of embryo transfer allows for fine tuning of procedures for patients thereby improving implantation outcomes.

### Example 2: Minimally Invasive Uterine Aspiration 24 Hours Ahead of Embryo Transfer Characterizes the Compromised RIF Uterine Microenvironment and is Predictive of Reproductive Outcome

Objective: Repeat implantation failure (RIF) is particularly challenging to treat, resulting in limited success, even when adequate preparation of the endometrium is established and a transfer is performed with a high grade euploid blastocyst. The objective of this study was to utilize a multidisciplinary approach to decipher the complexity of RIF through investigations of the maternal molecular components ahead of an embryo transfer.

Materials and Methods: Patients were recruited with IRB consent 24 hours prior to a programmed frozen embryo transfer (FET) with a euploid blastocyst. Uterine secretions were collected by gentle aspiration (˜2-50) under ultrasound guidance and grouped according to reproductive outcomes: Failed euploid FET (RIF patients, ≥3 prior IVF failures) and Positive live birth FET (maternally age-matched patients; mean 36.6±3.8 years). Total and small RNA (n=22) was isolated for sequencing on the NovaSEQ 6000 (Illumina). Reads were aligned to hg38 using GSNAP and analyzed with edgeR (FDR cutoff of 5%, P<0.01). Metabolite analysis (n=20) was performed by UHPLS-MS (Thermo) using MassMatrix and Maven (Princeton Univ.). Proteomic analysis (n=6) involved FASP digestion and LC-MS/MS, with protein identifications generated by Mascot (v2.6) and Scaffold (v4.8.9) (α of 0.05; fold change >1.5 or <0.5).

Results: A unique uterine microenvironment was observed for RIF patients and negative implantation outcomes 24 hours prior to an embryo transfer (P<0.05). An interplay of several biological processes were evident in RIF failed aspirates with focused interest of 13 significantly reduced transcripts, 7 significantly increased maternal miRNAs, 12 significantly decreased amino acids and 16 proteins of significantly altered abundance (P<0.05). See Tables 3 and 4. Specific examples included: decreased expression of PLA2G4D (P<0.0001), which regulates the eicosanoid pathway, thereby impacting downstream synthesis of prostaglandins like PGE2; decreased expression of TET1 (P<0.0001), an epigenetic regulator required for DNA methylation; increased expression of miR-17, a known negative regulator of VEGFA, required for successful implantation (P<0.01); decreased quantities of arginine, essential for blastocyst activation and trophectoderm motility (P<0.05); and an increased abundance of SERPING1, a protein associated with inflammation, which regulates complement activation (P<0.05).

TABLE 3
proteins having decreased expression
associated with negative implantation

| | Negative |
|---|---|
| Protein | Implantation |
| SOD1 | Decreased Expression |
| PRDX6 | Decreased Expression |
| PLA2G4D | Decreased Expression |
| TET1 | Decreased Expression |

TABLE 4
proteins having increased expression
associated with negative implantation

| | Negative |
|---|---|
| Protein | Implantation |
| ITIH4 | Increased Expression |
| LTF | Increased Expression |
| SERPING1 | Increased Expression |
| GC | Increased Expression |
| CFH | Increased Expression |
| CP | Increased Expression |
| GGT1 | Increased Expression |
| THSD4 | Increased Expression |
| ANPEP | Increased Expression |
| COL6A1 | Increased Expression |
| PROM1 | Increased Expression |

Conclusion: Analysis of uterine secretions 24 hours prior to FET, allowed for an in-depth molecular characterization of the compromised RIF uterine microenvironment and is predictive of reproductive outcome. The negative influence on key miRNAs and gene transcription levels, in addition to altered amino acid and protein concentrations, were all identified as critical contributors to poor RIF outcomes. These findings facilitate more effective clinical interventions for this difficult patent population.

What is claimed is:

1. A method of predicting negative pregnancy outcome in fertility treatment, the method comprising:
contacting uterine endometrial secretions from a patient with a kit that comprises a solid-state substrate functionalized to identify one or more, or at least two, markers associated with a hostile endometrial environment selected from the group consisting of proteins, metabolites, or miRNAs, and
determining the secretome profile of the uterine endometrial secretions to ascertain an increase or decrease in the presence of markers associated with a hostile endometrial environment,
wherein the increase or decrease in the presence of one or more markers associated with a hostile endometrial environment, relative to the secretome profile of uterine endometrial secretions of a successful pregnancy outcome, predicts negative pregnancy outcome.

2. The method of claim 1, wherein the marker is a protein selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment.

3. The method of claim 1, wherein the marker is a protein selected from the group consisting of SOD1, PRDX6, PLA2G4D, and TET1, wherein decreased expression of the protein is associated with a hostile endometrial environment.

4. The method of claim 1, wherein the marker is arginine, wherein decreased levels of arginine is associated with a hostile endometrial environment.

5. The method of claim 1, wherein the marker is a microRNA selected from the group consisting of hsa-miR-891a, hsa-miR-522, hsa-miR-198, and hsa-miR-365, and wherein decreased presence is associated with a hostile endometrial environment.

6. The method of claim 1, wherein the marker is a microRNA selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and wherein increased presence is associated with a hostile endometrial environment.

7. The method of claim 1, wherein the marker is a metabolite selected from the group consisting of urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline, and wherein decreased presence is associated with a hostile endometrial environment.

8. A kit comprising a solid-state substrate functionalized to identify one or more, or at least two, markers associated with a hostile endometrial environment selected from the group consisting of proteins, metabolites, and miRNAs.

9. The kit of claim 8, wherein the marker is a protein selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment.

10. The kit of claim 8, wherein the marker is a protein selected from the group consisting of SOD1, PRDX6, PLA2G4D, and TET1, wherein decreased expression of the protein is associated with a hostile endometrial environment.

11. The kit of claim 8, wherein the marker is arginine, wherein decreased levels of arginine is associated with a hostile endometrial environment.

12. The kit of claim 8, wherein the marker is a microRNA selected from the group consisting of hsa-miR-891a, hsa-miR-522, hsa-miR-198, and hsa-miR-365, and wherein decreased presence is associated with a hostile endometrial environment.

13. The kit of claim 8, wherein the marker is a microRNA selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and wherein increased presence is associated with a hostile endometrial environment.

14. The kit of claim 8, wherein the marker is a metabolite selected from the group consisting of urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline, and wherein decreased presence is associated with a hostile endometrial environment.

15. A system for enhancing the pregnancy success rate of fertility treatment, comprising predicting a negative pregnancy outcome in a patient undergoing fertility treatment prior to frozen embryo transfer or intrauterine insemination, wherein the predicting comprises determining the secretome profile of the patient's uterine endometrial secretions to ascertain an increase or decrease in the presence of markers associated with a hostile endometrial environment,
wherein the increase or decrease in the presence of one or more markers associated with a hostile endometrial environment, relative to the secretome profile of uterine endometrial secretions of a successful pregnancy outcome, predicts negative pregnancy outcome in the patient.

16. The system of claim 15, wherein the step of determining the secretome profile of uterine endometrial secretions comprises contacting uterine endometrial secretions from a patient with a kit that comprises a solid-state substrate functionalized to identify one or more, or at least two, markers associated with a hostile endometrial environment selected from the group consisting of proteins, metabolites, or miRNAs, and determining the secretome profile of the uterine endometrial secretions to ascertain an increase or decrease in the presence of markers associated with a hostile endometrial environment.

17. The system of claim 15, wherein the marker is a protein selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment.

18. The system of claim 15, wherein the marker is a protein selected from the group consisting of SOD1, PRDX6, PLA2G4D, and TET1, wherein decreased expression of the protein is associated with a hostile endometrial environment.

19. The system of claim 15, wherein the marker is arginine, wherein decreased levels of arginine is associated with a hostile endometrial environment.

20. The system of claim 15, wherein the marker is a microRNA selected from the group consisting of hsa-miR-891a, hsa-miR-522, hsa-miR-198, and hsa-miR-365, and wherein decreased presence of the microRNA is associated with a hostile endometrial environment.

21. The system of claim 15, wherein the marker is a microRNA selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and wherein increased presence of the microRNA is associated with a hostile endometrial environment.

22. The system of claim 15, wherein the marker is a metabolite selected from the group consisting of urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline, and wherein decreased presence of the metabolite is associated with a hostile endometrial environment.

23. The system of claim 15, wherein the secretome profile is obtained by mass spectroscopy.

24. The system of claim 15, wherein the secretome profile is obtained by data generated by Enzyme-Linked Immunosorbent Assay (ELISA).

25. The system of claim 15, wherein the secretome profile is obtained by qPCR.

26. An in vitro method of screening a fertility patient prior to frozen embryo transfer or intrauterine insemination, the method comprising contacting uterine endometrial secretions from a patient with a kit that comprises a solid-state substrate functionalized to identify one or more, or at least two, markers associated with a hostile endometrial environment selected from the group consisting of proteins, metabolites, or miRNAs, and
determining the secretome profile of the uterine endometrial secretions to ascertain an increase or decrease in the presence of markers associated with a hostile endometrial environment,
wherein the increase or decrease in the presence of one or more markers associated with a hostile endometrial environment, relative to the secretome profile of uterine endometrial secretions of a successful pregnancy outcome, predicts negative pregnancy outcome.

27. The method of claim 26, wherein the marker is a protein selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment.

28. The method of claim 26, wherein the marker is a protein selected from the group consisting of SOD1, PRDX6, PLA2G4D, and TET1, wherein decreased expression of the protein is associated with a hostile endometrial environment.

29. The method of claim 26, wherein the marker is arginine, wherein decreased levels of arginine is associated with a hostile endometrial environment.

30. The method of claim 26, wherein the marker is a microRNA selected from the group consisting of hsa-miR-891a, hsa-miR-522, hsa-miR-198, and hsa-miR-365, and wherein decreased presence is associated with a hostile endometrial environment.

31. The method of claim 26, wherein the marker is a microRNA selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and wherein increased presence is associated with a hostile endometrial environment.

32. The method of claim 26, wherein the marker is a metabolite selected from the group consisting of urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline, and wherein decreased presence is associated with a hostile endometrial environment.

33. A method of predicting implantation failure of a candidate embryo, the method comprising:
contacting uterine endometrial secretions from a patient with a kit that comprises a solid-state substrate functionalized to identify one or more, or at least two, markers associated with a hostile endometrial environment selected from the group consisting of proteins, metabolites, or miRNAs, and
determining the secretome profile of the uterine endometrial secretions to ascertain an increase or decrease in the presence of markers associated with a hostile endometrial environment,
wherein the increase or decrease in the presence of one or more markers associated with a hostile endometrial environment, relative to the secretome profile of uterine endometrial secretions of a successful pregnancy outcome, predicts embryo implantation failure.

34. The method of claim 33, wherein the marker is a protein selected from the group consisting of IL-6, IL-8, VEGF, Mucin-1, Mucin-16, Mucin-5B, Mucin-5AC, IgGFc-binding protein, Carbonic anhydrase 1, Cystatin-C, ITIH4, LTF, SERPING1, GC, CFH, FFT1, THSD4, ANPEP, COL6A1, PROM1, and PLG, wherein increased expression of the protein is associated with a hostile endometrial environment.

35. The method of claim 33, wherein the marker is a protein selected from the group consisting of SOD1, PRDX6, PLA2G4D, and TET1, wherein decreased expression of the protein is associated with a hostile endometrial environment.

36. The method of claim 33, wherein the marker is arginine, wherein decreased levels of arginine is associated with a hostile endometrial environment.

37. The method of claim 33, wherein the marker is a microRNA selected from the group consisting of hsa-miR-891a, hsa-miR-522, hsa-miR-198, and hsa-miR-365, and wherein decreased presence is associated with a hostile endometrial environment.

38. The method of claim 33, wherein the marker is a microRNA selected from the group consisting of hsa-miR-135a, hsa-miR-17, hsa-miR-10b, hsa-miR-126, hsa-miR-155, hsa-miR-19a, hsa-miR-150, hsa-miR-200c, hsa-miR-224, hsa-miR-140, hsa-miR-222, hsa-miR-31, hsa-miR-454, hsa-miR-106c, and wherein increased presence is associated with a hostile endometrial environment.

39. The method of claim 33, wherein the marker is a metabolite selected from the group consisting of urate, xanthine, docosahexaenoic acid, fumarate, cysteine, citrate, putrescine, proline, orthophosphate, leucine/isoleucine, hypoxanthine, heptanoic acid, alanine, adenosine, 8z-11z-14z-icosatrienoic acid, 8z-11z-14z-17z-icosapentaenoic acid, and 5-oxoproline, and wherein decreased presence is associated with a hostile endometrial environment.

40. A method of treating female infertility comprising:
(a) ascertaining miR-17 levels in the uterine endometrial secretions from a patient,
(b) treating a patient having an increase in miR-17 levels in the uterine endometrial secretions relative to a miR-17 profile indicative of a successful pregnancy outcome with recombinant human VEGF-A; and
(c) performing frozen embryo transfer or intrauterine insemination.

41. The method of claim 40, wherein (a) is performed at least 24 hours prior to (c).
